(19) United States
(12) Patent Application Publication
(10) Pub. No.: US 2025/0326807 A1
(43) Pub. Date: Oct. 23, 2025

(54) CALPROTECTIN BINDING PEPTIDES

(71) Applicant: Buehlmann Laboratories AG, Schoenenbuch (CH)

(72) Inventors: Cristina Diaz Perlas, Barcelona (ES); LIuc Farrera Soler, Genève (CH); Christian-Benedikt Gerhold, Efringen-Kirchen (DE); Dmitrii Guschin, Efringen-Kirchen (DE); Christian Heinis, Bern (CH); Kelvin Lau, Les Acacias (CH); Florence Pojer, St. Sulpice VD (CH); Benjamin Ricken, Schopfheim (DE)

(21) Appl. No.: 18/860,586

(22) PCT Filed: Apr. 26, 2023
(86) PCT No.: PCT/EP2023/061044

§ 371 Date: Oct. 25, 2024

(30) Foreign Application Priority Data
Apr. 28, 2022 (EP) 22170615.3

(51) Int. Cl. C07K 14/47; C07K 1/14; C07K 7/06; C07K 14/00; G01N 33/543; G01N 33/68

(52) U.S. Cl. CPC C07K 14/4728; C07K 1/14; C07K 7/06; C07K 14/001; G01N 33/543; G01N 33/68; G01N 2333/4727

(57) ABSTRACT

The invention relates to peptides capable of binding calprotectin. The invention is further directed to methods of detecting and purifying calprotectin.

### FIELD OF THE INVENTION

The invention relates to synthetic peptide ligands capable of binding calprotectin.

### BACKGROUND

Calprotectin (CP) is a cytoplasmic protein expressed in various myeloid cell types, such as neutrophils, monocytes, and macrophages. In neutrophils, calprotectin is constitutively expressed and constitutes approximately 40% of the total cytoplasmic protein, while in epithelial cells and keratinocytes, calprotectin expression can be induced.

Calprotectin consists of two polypeptide chains, Mrp8 (synonyms: S100A8, Calgranulin A) and Mrp14 (synonyms: S100A9, Calgranulin B), that form a stable dimer. In the presence of about 150 μM Ca²⁺, two Mrp8/Mrp14 heterodimers can form a heterotetramer, which plays an important role in nutritional immunity as a sequestration complex for divalent cations such as Zn²⁺, leading to starvation of microbes during inflammation procedures (Zygiel E M, Nolan E M. Transition Metal Sequestration by the Host-Defense Protein Calprotectin (2018). Annu. Rev. Biochem. 87:621-43).

Due to its release at inflammation sites, calprotectin is considered to be an alarmin and is frequently used as a biomarker to monitor inflammatory processes. For example, fecal calprotectin is currently the gold standard to diagnose and monitor inflammatory bowel diseases (IBD), such as Crohn's disease (CD) and Ulcerative Colitis (UC) (Konikoff MR, Denson L A. Role of Fecal Calprotectin as a Biomarker of Intestinal Inflammation in Inflammatory Bowel Disease (2006). Inflamm. Bowel Dis. 12 (6): 524-34).

Moreover, serum CP is validated as a biomarker to monitor various (chronic) inflammatory diseases, e.g., rheumatoid arthritis (Austermann J et al. S100 proteins in rheumatic diseases (2018). Nat. Rev. Rheumatol. 14:528-541; Ometto F et al. Calprotectin in rheumatic diseases (2017). Exp. Biol. Med. 242:859-873).

Existing calprotectin assays are based on antibodies and therefore face the challenges commonly associated with antibody affinity reagents, in particular limited shelf life, high production costs, high batch variability and non-homogenous immobilization.

In contrast to antibodies, synthetic peptide ligands consisting of several dozens to a few hundred amino acids are suitable for mass production, show little to no batch-to-batch variability and are more stable during storage. However, such ligands have not been described yet for calprotectin.

### OBJECTIVE PROBLEM TO BE SOLVED

There is therefore a need in the art to provide high affinity peptide ligands capable of binding calprotectin. These could be used not only for the detection of calprotectin, but also for its purification and for medical purposes.

### SUMMARY OF THE INVENTION

In one aspect, the invention relates to a peptide capable of binding calprotectin, the peptide comprising the sequence ΦZΨΣXΘΘΘΩ, wherein
Φ is L-phenylalanine, L-tryptophan or an analogue thereof,
X and Z represent an α-amino acid with L-configuration,
Ψ represents a nonpolar, aliphatic L-amino acid,
Σ is L-tyrosine, L-tryptophan, L-phenylalanine or an aromatic L-amino acid,
Θ represents an α-amino acid,
Ω represents a hydrophobic L-amino acid and
   wherein Φ at each occurrence, Z at each occurrence, X at each occurrence, Ψ at each occurrence, Σ at each occurrence, Θ at each occurrence and Ω at each occurrence, are selected independently from each other.

In another aspect, the invention relates to a method for detecting calprotectin in a sample, the method comprising
a) providing a sample comprising calprotectin;
b) contacting the sample with the peptide according to the invention and allowing the peptide form a complex with the calprotectin;
c) detecting the complex formed in step b).

In another aspect, the invention relates to a kit for detecting calprotectin in a sample, the kit comprising the peptide according to the invention.

In another aspect, the invention relates to a method of purifying calprotectin, the method comprising purifying the calprotectin using a peptide according to the invention.

In another aspect, the invention relates to a pharmaceutical composition comprising the peptide according to the invention.

In another aspect, the invention relates to use of a peptide according to the invention for tagging a protein of interest.

### BRIEF DESCRIPTION OF THE FIGURES

FIG. 1 shows the fluorescence polarization results of calprotectin binding peptides derived from phage display. Selected peptide sequences from three different phage-display libraries were enriched after biopanning with human recombinant calprotectin. These peptides were then synthesized and cross-linked in between cysteines. Fluorescence polarization experiments reveal affinities to human recombinant calprotectin in the nanomolar range.

FIG. 2 shows the characterization of a calprotectin binding peptide having the sequence RSPESVAFPMFQSHWYSG (peptide 3). Different cross-linking isomers were synthesized, purified and tested in fluorescence polarization. A) Isomer 1 with cross-linked neighboring cysteines showed the most significant increase in fluorescence polarization and a K_{D} of about 24±10 nM. B) Further characterization of the requirement for cross-linking was achieved by selective replacement of cysteines with serines. Isomers 1a and 1b with only one cross-link of neighboring cysteines each were synthesized as well as a fully linear peptide comprising only serines instead of cysteines. All three peptides exhibited comparable affinities to recombinant calprotectin suggesting that peptide 3 binds calprotectin in an elongated linear conformation.

FIG. 3 shows the alanine scan of a calprotectin binding peptide to reveal amino acids that are involved in binding. A) The linear peptide was synthesized with replacement of selected amino acids with alanine or glycine. Subsequently, slightly modified linear peptides derived from peptide 3 were tested with respect to their affinity to recombinant calprotectin using B) surface plasmon resonance (SPR) and C) fluorescence polarization (FP). Most replacements of amino acids did not change the binding affinity significantly. The replacement of F8, M10, F11, H14 and Y16 with alanines showed a significant decrease in affinity in SPR and FP, suggesting that these amino acids are mostly responsible for the interaction between the peptide 3 and human calprotectin.

FIG. 4 shows the X-ray structure of the peptide in complex with calprotectin highlighting the binding interface between peptide and calprotectin tetramer. A) Co-crystallization of calprotectin with the peptide reveals that two peptides 3 can bind to one calprotectin tetramer. One peptide exhibits contacts to two S100A8 (Mrp-8) and one S100A9 (Mrp-14). Thus, the complete calprotectin binding groove for peptide 3 requires alignment of two S100A8 molecules found only in the tetramer, which explains the affinity of peptide 3 to the tetrameric form. B) Binding groove zoom-in with peptide 3 in a stick model. Amino acids that are important for binding affinity according to the alanine scan are underlined. C) Valence structural formula of peptide 3 with interactions to calprotectin.

FIG. 5 shows the suitability of calprotectin binding peptides in an ELISA. A) Example set-up of a calprotectin recognizing enzyme-linked immunoabsorbent assay (ELISA). calprotectin binding antibodies are coated on a plate and immobilize calprotectin from a sample. The detection of calprotectin is performed by incubation with biotinylated calprotectin binding peptide 3 in complex with streptavidin conjugated horse radish peroxidase (Step-HRP) for tetramethylbenzidine (TMB) transition. B) Comparison between C- and N-terminal biotinylation of peptide 3 in an ELISA set-up. The calprotectin concentration dependent signal is more pronounced when biotin is added on the C-terminus. C) Time resolved increase in signal dependent on calprotectin concentration in an ELISA showing that high sensitivities can be achieved when incubation times of 60 minutes are used. D) Determination of background signals in the ELISA set up. Low background signals are achieved without the use of peptide or in absence of calprotectin. The C-terminal biotinylation of peptide number 3 is favourable for better signal to noise ratios.

FIG. 6 shows calprotectin binding peptide in a lateral flow-immunoassay set-up. A) Dipstick experiments with coated calprotectin as control dot and rabbit polyclonal anti-calprotectin-antibodies as test dot. Gold nanoparticles are conjugated with peptide 3 and added to the sample well as well as calprotectin of different concentrations. Test dot intensity increased with rising calprotectin concentrations in the specimen, suggesting the possibility that a quantitative measurement of calprotectin is possible with this set-up based on either test dot intensities or ratios of test over control dots. B) Full lateral flow set-up with lined anti calprotectin antibodies on the test line and recombinant calprotectin on the control line. This set-up is compatible with the detection of 950 ng/mL calprotectin from buffer, but also serum or blood samples. C) Different linear calibration curves based on T/C ratios (test line over control line intensity ratio) with buffer or serum spiked with recombinant calprotectin or native calprotectin derived from granulocytes. D) Comparison of T/C ratios patient sera of 18 rheumatoid arthritis (RA) patients with sera of 9 healthy donors illustrates that the peptide based lateral flow may have diagnostic value for inflammatory diseases with increased calprotectin concentrations. E) A receiver operating curve analysis based on these data results in an area under the curve of 0.858.

FIG. 7 shows the L-Alanine-, D-alanine- and β-alanine scan of Peptide 3. The K_{D} values represented in the graphic are the average of K_{D} obtained by kinetic and steady-state analysis in SPR. Compounds which gave a signal of less than 2 response units (which is much lower than the typically observed 40 unit-change found for peptide 3) at the highest concentration tested (500 nM) were assigned a K_{D} value >10⁻⁵ M. The green dashed line indicates the K_{D} of peptide 3. The SPR data correlates well with affinity measurements in a fluorescence polarization (FP) based assay: 95% of the compounds that showed binding in SPR (K_{D} value smaller than 10⁻⁵ M) showed binding in FP by an increase in fluorescence anisotropy of at least 4 units at 330 nM of calprotectin.

FIG. 8 shows that mutation of amino acids Phe8, Met10, Phe11, His14 and Tyr 16 in peptide 3 that were found to be most important for binding in the L-alanine scan. All positions were mutated to at least one aromatic amino acid (Phe, Tyr or Trp), Glu (as representative of a charged amino acid), Gln (as representative of a polar amino acid) and IIe (as representative of an aliphatic amino acid). A few additional mutations were studied. The K_{D} values represented in the graphic are the average of K_{D} obtained by kinetic and steady-state analysis in SPR. Compounds which gave a signal of less than 2 response units (which is much lower than the typically observed 40 unit-change found for peptide 3) at the highest concentration tested (500 nM) were assigned a K_{D} value >10⁻⁵ M. The green dashed line indicated the K_{D} of peptide 3.

### DETAILED DESCRIPTION

In one aspect, the invention relates to a peptide capable of binding calprotectin, the peptide comprising the sequence ΦZΨΣXΘΘΘΩ, wherein
Φ is L-phenylalanine, L-tryptophan or an analogue thereof,
X and Z represent an α-amino acid with L-configuration,
Ψ represents a nonpolar, aliphatic L-amino acid,
Σ is L-tyrosine, L-tryptophan, L-phenylalanine or an aromatic L-amino acid,
Θ represents an α-amino acid,
Ω represents a hydrophobic L-amino acid and
   wherein Φ at each occurrence, Z at each occurrence, X at each occurrence, Ψ at each occurrence, Σ at each occurrence, Θ at each occurrence and Ω at each occurrence, are selected independently from each other.

If a variable is herein defined as “being x, x or x”, this wording is considered equivalent to the variable “is selected from the group consisting of x, x and x”.

As used herein, the term “peptide” refers to an amino acid chain having a maximum length of 200 amino acids.

As used herein, the term “amino acid” refers to organic compounds containing amino and carboxylate functional groups and, optionally, one or more side chains that may also carry functional groups. In amino acids that have a carbon chain attached to the a-carbon (such as lysine) the carbons are labeled α, β, γ, δ, and so on. In some amino acids, the amine group may be attached, for instance, to the α-, β- or γ-carbon, and these are therefore referred to as α-, β- or γ-amino acids, respectively.

Proteinogenic amino acids, also termed naturally occurring amino acids, are amino acids that are biosynthetically incorporated into proteins during translation. Other than the amino acids encoded by naturally occurring base triplets, proteinogenic amino acids also encompass selenocysteine and pyrrolysine.

Non-proteinogenic amino acids are amino acids that are non-coded but can nonetheless be integrated into peptides. The person skilled in the art is aware which compounds fall under the definition of non-proteinogenic amino acids. Non-proteinogenic amino acids include, for example, all-S,all-E-3-amino-9-methoxy-2,6,8-trimethyl-10-phenyldeca-4,6-dienoic acid (ADDA), B-alanine, 4-aminobenzoic acid, gamma-aminobutyric acid, S-aminoethyl-L-cysteine, 2-aminoisobutyric acid, aminolevulinic acid, azetidine-2-carboxylic acid, canaline, canavanine, carboxyglutamic acid, chloroalanine, citrulline, cysteine, dehydroalanine, diaminopimelic acid, dihydroxyphenylglycine, enduracididine, homocysteine, homoserine, 4-hydroxyphenylglycine, hydroxyproline, hypusine, lanthionine, B-leucine, mimosine, norleucine, norvaline, ornithine, penicillamine, plakohypaphorine, pyroglutamic acid, quisqualic acid, sarcosine, theanine, tranexamic acid, tricholomic acid and 3,4-dihydroxyphenylalanine (L-DOPA).

As used herein, the term “(amino acid) derivatives” is defined as proteinogenic or non-proteinogenic amino acids modified by the addition or replacement of individual functional groups.

Amino acids are generally classified by the chemical properties of their side-chain, i.e., a branch from the parent structure of the amino acid. Aromatic amino acids and aliphatic amino acids are terms known in the art. Aromatic amino acids comprise at least one aromatic ring. Aromatic amino acids include phenylalanine, tryptophane, tyrosine and derivatives thereof. Aliphatic amino acids comprise at least one aliphatic side chain or a side chain displaying properties similar to an aliphatic side chain, i.e. are nonpolar and hydrophobic. Aliphatic amino acids include alanine, leucine, isoleucine, norleucine, proline, valine, methionine and derivatives thereof. Hydrophobic amino acids include tyrosine, phenylalanine, tryptophan and isoleucine.

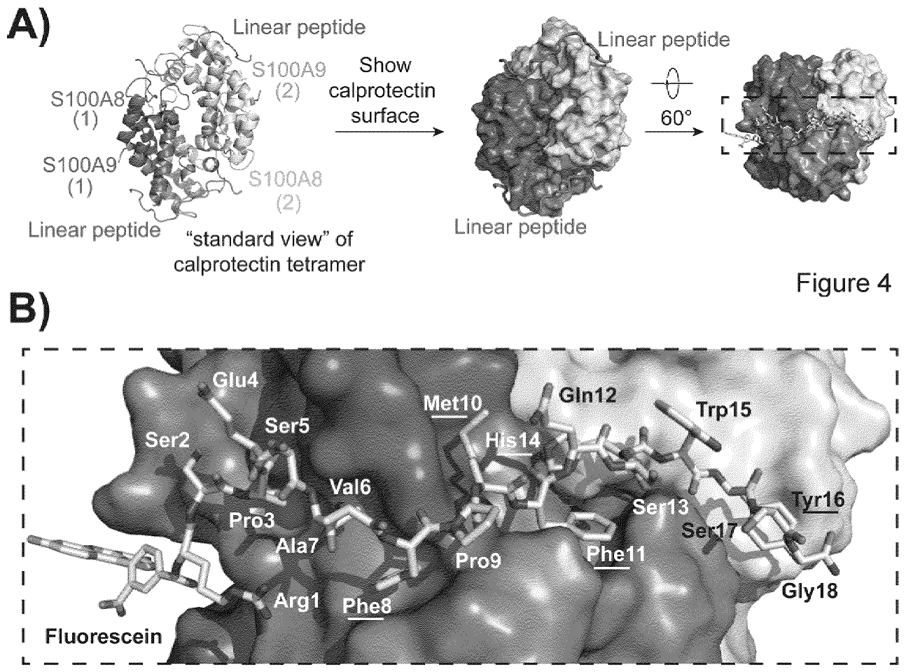

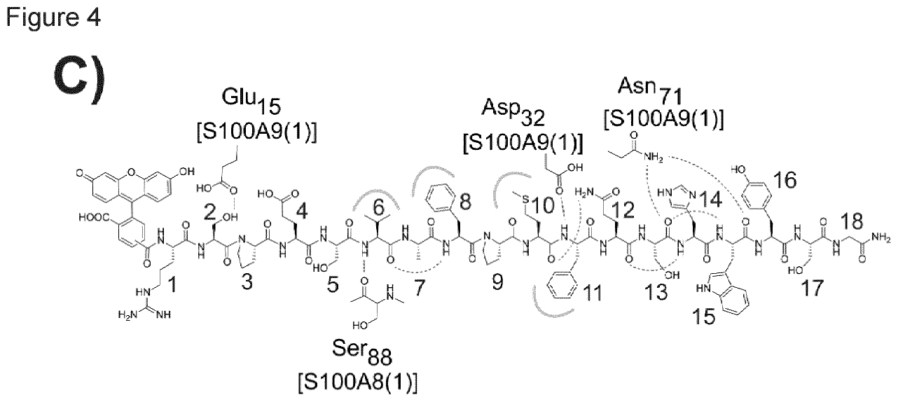

The present inventors have surprisingly identified the sequence ΦZΨΣXΘΘΘΩ, wherein
Φ is L-phenylalanine, L-tryptophan or an analogue thereof,
X and Z represent an α-amino acid with L-configuration,
Ψ represents a nonpolar, aliphatic L-amino acid,
Σ is L-tyrosine, L-tryptophan, L-phenylalanine or an aromatic L-amino acid,
Θ represents an α-amino acid,
Ω represents a hydrophobic L-amino acid and
   wherein Φ at each occurrence, X at each occurrence, Z at each occurrence, Ψ at each occurrence, Σ at each occurrence, Θ at each occurrence and Ω at each occurrence, are selected independently from each other, as a peptide capable of binding native, tetrameric calprotectin with high affinity. Advantageously, calprotectin dimers are bound with considerably lower affinity, because the peptides according to the invention bind an interface of Mrp8 and Mrp14 that is only fully formed upon tetramerization (FIG. 4). Consequently, the peptides according to the invention are particularly well suited for recognizing, detecting and purifying calprotectin in its naturally occurring tetrameric form.

In one embodiment, the peptides according to the invention have an equilibrium dissociation constant K_{D} of between 1 pM and 750 nM. In a preferred embodiment, K_{D} is below 200 nM, more preferably below 50 nM. Capability of binding calprotectin of a given peptide can be assessed by assays known in the art, for example by surface plasmon resonance, fluorescence polarization or bilayer interferometry.

In one embodiment, Ψ represents L-methionine, L-leucine, L-isoleucine or L-norleucine. In another embodiment that can be combined with the previous embodiment, Z represents L-proline or L-alanine.

In another embodiment, the peptide according to the invention fulfills at least one of the following conditions:
(1) Φ is L-phenylalanine or a derivative thereof;
(2) Z is L-proline or a derivative thereof;
(3) Ψ is L-methionine or a derivative thereof;
(4) Σ is L-phenylalanine or a derivative thereof;
(5) Ω is L-tyrosine or a derivative thereof.

In a preferred embodiment, the peptide fulfills condition (1) and at least one of conditions (2), (3), (4) or (5). In another embodiment, the peptide fulfills condition (2) and at least one of conditions (1), (3), (4) or (5). In another embodiment, the peptide fulfills condition (3) and at least one of conditions (1), (2), (4) or (5). In another preferred embodiment, the peptide fulfills conditions (1) and (2). In another preferred embodiment, the peptide fulfills conditions (1), (2) and at least one of (3), (4) or (5). In another preferred embodiment, the peptide fulfills conditions (1), (2), (3) and at least one of (4) or (5). In a particularly preferred embodiment, the peptide fulfills all five conditions.

In another preferred embodiment, the peptide comprises the sequence FPLFQΘXΘY, FPIFQΘXΘY, FP(NIe)FQΘXΘY, FPLFQΘXΘF, WPLFQΘXΘY, FPIFQΘXΘF, FP(NIe)FQΘXΘF, WPIFQΘXΘY, WP(NIe)FQΘXΘY, WPLFQΘXΘF, WPIFQΘXΘF, WP(NIe)FQΘXΘF or FZMFXΘHΘY.

In a particularly preferred embodiment, the peptide comprises the sequence FPLFQΘXΘY, FPIFQΘXΘY, FP(NIe)FQΘXΘY, FPLFQΘXΘF or WPLFQΘXΘY, most preferably FPLFQΘXΘY.

The peptides according to the invention preferably consist of 9 to 30 amino acids, more preferably 15 to 20 amino acids, most preferably 18 amino acids. Their small size, compared to antibodies, facilitates chemical synthesis, leads to better tissue penetration and higher resistance to protease cleavage and inactivation and extends the peptides' half-life both in vivo and in vitro. In a particularly preferred embodiment, the peptide has or consists of the sequence RCPECVAFPMFQCHWYCG or RSPESVAFPMFQSHWYSG.

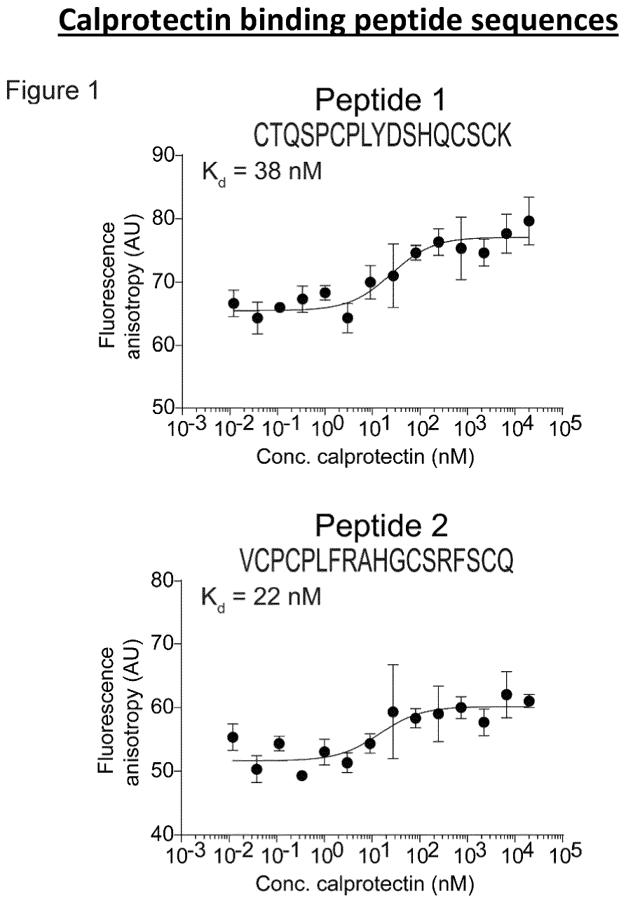

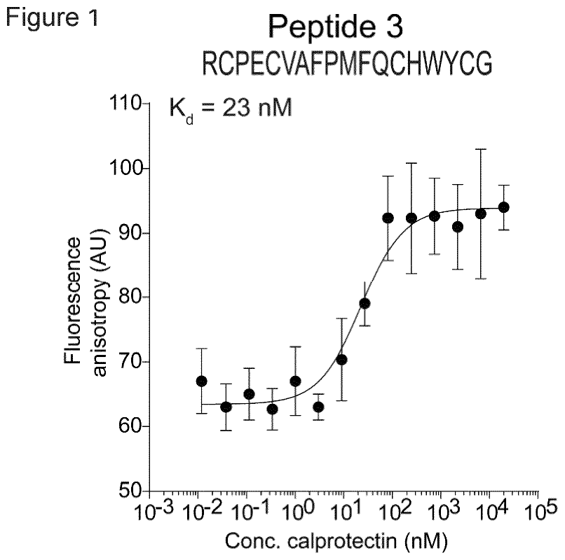

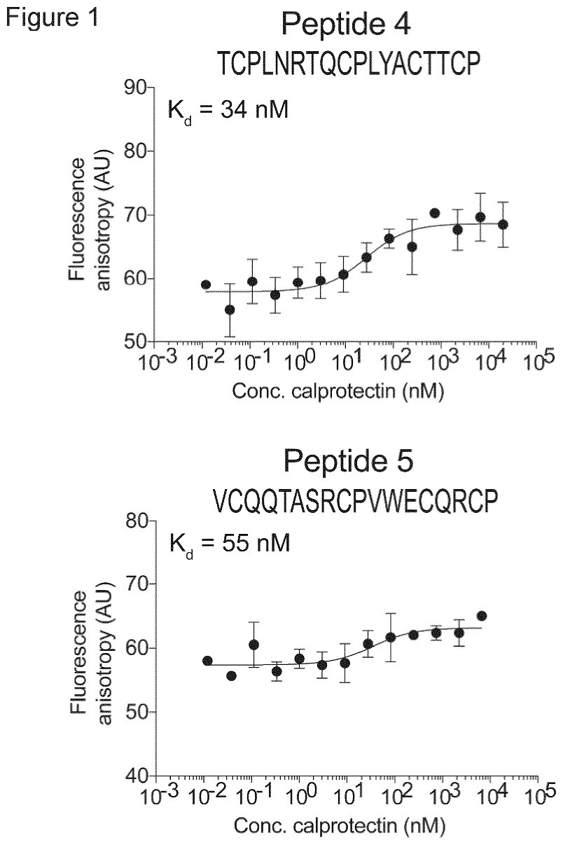

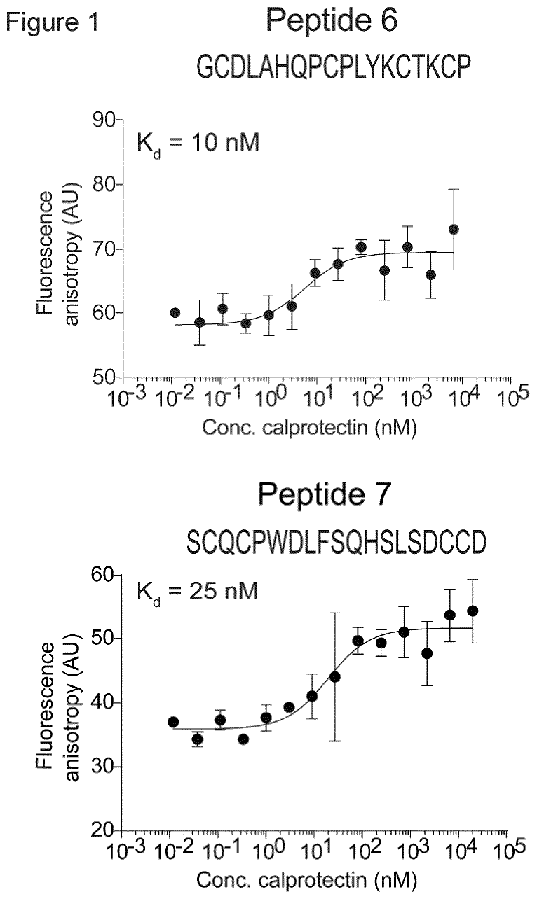

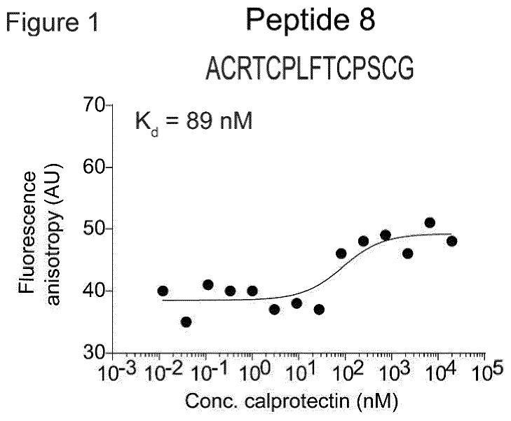

In another particularly preferred embodiment, the peptide capable of binding calprotectin is selected from the group consisting of CTQSPCPLYDSHQCSCK, VCPCPLFRAHGCSRFSCQ, CQCPWDLFSQHSLSDCCD, WCTQSPCPLYDSHQCSCK, TCPLNRTQCPLYACTTCP, GCDLAHQPCPLYKCTKCP, VCQQTASRCPVWECQRCP, ACRTCPLFTCPSCG, RCPECVAFPMFQCHWYCG, RSPESVAFPMFQSHWYSG or SCQCPWDLFSQHSLSDCCD. These peptides have been shown to have excellent binding affinity to calprotectin (FIG. 1).

In the peptides according to the invention, cysteine residues may be crosslinked with each other, so that each peptide comprises two cyclic structures. Such peptides are also called bicyclic peptides. Thus, cysteine containing peptides according to the invention are preferably bicyclic peptides. Because of the presence of one or more disulfide bonds within the peptide, bicyclic peptides are conformationally restrained, leading to a relatively small entropy cost upon binding and thus good binding affinity and specificity. Unlike antibodies, bicyclic peptides may also penetrate the blood-brain barrier.

In yet another aspect, the invention relates to a method of detecting calprotectin in a sample, the method comprising
a) providing a sample comprising calprotectin;
b) contacting the sample with the peptide according to the invention and allowing the peptide to form a complex with the calprotectin;
c) detecting the complex formed in step b).

In a preferred embodiment, the sample is a biological sample such as blood sample, a serum sample, a plasma sample, a saliva sample, a urine sample or a stool sample. Methods for the detection of calprotectin in a biological sample are useful for monitoring inflammatory processes. The sample may be further purified, stabilized, diluted or otherwise processed in order to facilitate the detection process and stabilize the calprotectin in the sample.

According to the methods of the invention, the sample is subsequently contacted with a peptide according to the invention and the peptide is allowed to form a complex with the calprotectin. The step of contacting the sample with the peptide may take any form suitable for bringing the sample and the peptide into contact. For example, the peptide may be added directly to the sample or the sample may be added to a container containing the peptide. In the latter embodiment, the peptide may be immobilized on a solid support or a stationary phase.

In one embodiment, the peptide is detectably labelled. The term “label” as used herein refers to any entity that can be attached or complexed to a peptide in order to simplify detection of said peptide. Preferable labels used according to the invention include nanoparticles, e.g., gold nanoparticles, proteins, e.g. streptavidin, enzymes, e.g., horseradish peroxidase, dyes, e.g., luminescent or fluorescent dyes, and small molecules, e.g., biotin. In a preferred embodiment, the peptide according to the invention is labeled with gold.

In the last step according to the methods of the invention, the complex comprising calprotectin and the peptide is detected. Any detection method known in the art may be used. The choice of detection method may depend on the label with which the peptide is labelled. Detection methods useful for application in the methods of the invention include optical readouts, absorption, UV/VIS spectroscopy, turbidimetry, nephelometry, light scattering, reflectometry, fluorescence, luminescence, chemiluminescence, surface plasmon resonance, amperometry, magnetometry, voltammetry, potentiometry, conductometry, coulometry, polarography, gravimetry and cantilevers.

In yet another aspect, the invention relates to kits for detecting calprotectin in a sample, wherein the kit comprises a peptide according to the invention. The kits according to the invention may comprise means to perform the methods for detecting calprotectin according to the invention. In a preferred embodiment, the kit may be in the form of a lateral-flow immunoassay (LFI), wherein the peptide is detectably labelled with nanoparticles, e.g. cellulose, polystyrol or europium, preferably gold, and applied on a release pad or immobilized on a membrane.

In another preferred embodiment, the kit may be in the form of a particle enhanced turbidimetric immunoassay (PETIA), wherein the peptide is conjugated to nanoparticles. In another preferred embodiment, the kit may be in the form of an enzyme linked immunosorbent assay (ELISA), wherein the peptide is directly or indirectly linked to a detection enzyme, chemiluminescence or fluorescence marker.

The kits according to the invention may also comprise buffers, solutions and instructions to perform the methods of the invention.

In another aspect, the invention relates to methods of purifying calprotectin, the method comprising purifying the calprotectin using a peptide according to the invention. Because of their high affinity for calprotectin, the peptides according to the invention can be used to capture and purify calprotectin. For example, the methods of the invention may be used to purify calprotectin from granulocytes or inclusion bodies. In one embodiment, the peptides according to the invention are immobilized on a stationary phase. In one embodiment, the method comprises a step of contacting calprotectin with the peptides according to the invention.

In another aspect, the invention relates to pharmaceutical compositions comprising the peptides according to the invention.

In another aspect, the invention relates to use of the peptides according to the invention for tagging a protein of interest. The term “tagging” as used herein refers to covalently or non- covalently linking a peptide to a protein of interest. Proteins of interest may be tagged and subsequently purified using the peptide tag linked with calprotectin.

### EXAMPLES

### Example 1: Affinity Testing of Synthesized Peptides Derived from Phage Display Against Human Calprotectin

Recombinantly expressed fused calprotectin (His₆-linker-S100A9-linker-S100A8) (product code: B-RCAL, BÜHLMANN Laboratories AG Schönenbuch, Switzerland) was immobilized on magnetic beads by random biotinylation of amino groups and addition to streptavidin- or neutravidin-coated beads. The two types of beads were used alternatively to disfavour enrichment of streptavidin- or neutravidin-specific peptides. Neutravidin beads were prepared by reacting 6 mg of neutravidin (Pierce) with 10 mL of tosyl-activated magnetic beads (Dynal, M-280 from Invitrogen) according to the supplier's instructions.

Calprotectin was biotinylated by incubating 500 μL of calprotectin (10 μM) with 5 μL of EZ-Link™ Sulfo-NHS-LC-Biotin (Thermo Fisher Scientific) (10 mM, final conc. 200 μM, 20-fold molar excess) in 20 mM HEPES (pH 7.5), 150 mM NaCl and 2 mM CaCl₂. The reaction was incubated for 1 hr at room temperature. The protein was separated from the unreacted reagent using a PD-10 column (GE Healthcare). The sample was concentrated and stored at −80° C.

Biotinylated protein was immobilized on magnetic beads by incubation of protein and pre-washed beads in 200 μL washing buffer (10 mM Tris, pH 7.4, 150 mM NaCl, 10 mM MgCl₂, 2 mM CaCl₂) for 30 min at room temperature on a rotating wheel (10 rpm). Then, 3 μL of biotin (1 mM) was added to block the remaining positions on the beads for another 30 min. The beads were washed three times with 1 mL of washing buffer and resuspended in 400 μL washing buffer with 1% BSA and 0.1% Tween-20.

Phage-display libraries were generated as described (Kong et al. Generation of a Large Peptide Phage Display Library by Self-Ligation of Whole-Plasmid PCR Product ACS Chem. Biol. 2020). Library glycerol stock was inoculated in 0.5 L 2YT/tetracycline (100 μg/mL) culture. A sample was taken to calculate the initial phage titers. The culture was grown at 30° C. overnight with shaking (200 rpm). On the next day, the culture was pelleted at 4500 g at 4° C. and supernatant samples were taken. Phage precipitation was performed by adding 125 ml of cooled PEG/NaCl solution (20% PEG-6000 (w/v), 2.5 M NaCl), followed by incubation for 30 min on ice. Phages were then centrifuged at 6500 g for 45 min at 4° C. Then phage pellets were resuspended in 15 mL degassed reaction buffer (20 mM NH₄HCO₃, pH 8.0, 5 mM EDTA). Remaining cells were removed by centrifugation at 4500 g for 15 min at 4° C. Aliquots of phage were taken before and after the precipitation to calculate the phage titers.

The cysteine residues of the peptides were reduced by adding 1 mM TCEP for 30 min at 25° C. Phage precipitation was again performed by PEG/NaCl addition and the phage were resuspended in 18 mL of the degassed reaction buffer. For each linker, 4.5 mL of phage were taken and 30 μM to 40 μM of linker in 0.5 mL ACN was added. They were incubated at 30° C. for 1h and the phage were again precipitated by the addition of PEG/NaCl. The phage pellet was resuspended in 5 mL binding buffer (10 mM Tris, pH 7.4, 150 mM NaCl, 10 mM MgCl₂, 2 mM CaCl₂, 1% BSA and 0.1% Tween-20) and stored at 4° C. overnight.

For phage selection against calprotectin, the protein was immobilized on magnetic beads, as described previously. 5, 2.5, and 1 μg of target protein were immobilized on 20 μL of streptavidin beads (1^{st} and 3^{rd} round) or on 10 μL of neutravidin beads (2^{nd} round), respectively. Beads were then added to each modified phage and incubated for 30 min with rotation. Unbound phage was removed by washing the beads with washing buffer (with 0.1% Tween-20) for 8 times and washing buffer for 3 more times. The beads were resuspended in 100 μL glycine buffer (20 mM, pH 2.2) and incubated for 5 min to elute the phage. The solution was neutralized by adding 100 μL of Tris-Cl buffer (1 M, pH 8.0).

The eluted phages were added to 10 mL of E. coli TG1 cells at OD₆₀₀=0.4. After incubation at 37° C. for 30 min without shaking, the freshly infected bacteria were plated on 2YT/tetracycline (100 μg/mL) plates and grown overnight at 30° C. Bacterial cells of the colonies grown overnight were recovered in 2YT medium containing 20% glycerol, flash-frozen and stored at −80° C. until the next round of selection.

For the next rounds of selection, the scale for phage production was reduced to 25 mL per linker, and the rest of the solution volumes were adjusted accordingly. After the 3^{rd} round of selection, 24 clones per linker were sequenced by Sanger sequencing (Macrogen) and resulting sequences were grouped based on similarity.

Solid phase peptide synthesis (SPPS) based on these sequences was performed using Fmoc-chemistry, DMF as solvent and rink amide AM resin on a MultiPep RSi parallel peptide synthesizer (Intavis Inc.). Peptides were synthesized at a 25 μmol scale. Amino acids were coupled twice (2.5 eq.), using HATU (2.5 eq.), and NMM (4 eq.) and each coupling was performed at room temperature for 45 min. After the coupling reaction, 7 washing cycles with DMF were performed. N-terminal amines remaining free after coupling were capped using acetic anhydride (5% v/v) and lutidine (6% v/v) at RT for 30 minutes. Seven washing cycles were again performed. Fmoc groups were deprotected twice using piperidine (20% v/v) in DMF at room temperature for 5 min. Seven washing cycles were performed again. The N-terminal carboxyfluorescein moiety was incorporated manually with the addition of 5(6)-carboxyfluorescein (3 eq.), HATU (4 eq.) and DIEA (3 eq.) for 2×45 min. After, the resin was washed with DMF, piperidine (20% v/v), DMF and DCM.

Total cleavage of peptides was performed with a standard cleavage cocktail (90% TFA, 2.5% thioanisol, 2.5% H₂O, 2.5% 1.2-ethanedithiol, 2.5% phenol). 5 mL of cleavage cocktail were added to each peptide and incubated for 4 h while shaking. Peptide-containing solution was collected by vacuum filtration and peptides were then initially purified by cold ether precipitation. 50 mL of ice-cold diethyl ether were added to the peptides, incubated for 30 min at −20° C. and then centrifuged at 2700 g for 10 min. Peptide pellets were washed another time with 35 mL of diethyl ether and centrifuged again to remove remaining diethyl ether.

Linear peptides were purified with an HPLC system (Prep LC 2535 HPLC, Waters) using a preparative C18 reversed-phase column (Sunfire™ prep C18 OBD 10 μm, 100 Å, 19×250 mm, Waters) applying a flow rate of 20 mL/min and an appropriate linear gradient in 40 min (A: H₂O, 0.1% TFA; B: ACN, 0.1% TFA). Fractions containing the desired peptide were pooled together and lyophilized. This intermediate purification step was only performed for double-bridge peptides or when the crude mixture was too complex to perform the cyclization directly.

The purified peptide was dissolved in 10 mL of 30% v/v ACN and 70% v/v aqueous buffer (60 mM NH₄HCO₃, pH 8.0) and the cyclization reagent was added in ACN (3 eq., 100 μL).

The reaction mixture was incubated at 30° C. for 1 hr, and the completion of the reaction was assessed by LCMS. The reaction was stopped by addition of HCOOH (200 μL) and the cyclized peptide was lyophilized. The final purification was performed as before with a reversed-phase C18 column (X-bridge peptide BEH C18 5 μm, 300 Å, 10×250 mm, Waters) applying a flow rate of 6 mL/min and an appropriate linear gradient in 40 min. Fractions containing the desired peptide were lyophilized. The purity of the peptides was assessed by analysing around 20 μg of peptide by RP-HPLC (1260 HPLC system, Agilent) using a C18 column (ZORBAX 300SB-C18, 5 μm, 300 Å, 4.6×250 mm, Agilent). Peptides were run at a flow rate of 1 mL min⁻¹ with a linear gradient of 0-100% of solvent B over 15 min (A: 94.9% H₂O, 5% ACN and 0.1% TFA; B: 99.9% ACN and 0.1% TFA). The mass was determined by electrospray ionization mass spectrometry (ESI-MS) in positive ion mode on a single quadrupole liquid chromatography mass spectrometer (LCMS-2020, Shimadzu). In order to measure the affinity of selected and synthesized peptides in fluorescence polarization, calprotectin was serially diluted in 20 mM HEPES, 100 mM NaCl, 2 mM CaCl₂, pH 7.5, 1 mM DTT with 0.01% v/v Tween-20. 16 μL of protein were added to 4 μL of the fluorescent peptide (20 nM final concentration) in 96-well microtiter plates (black, half-area). Fluorescence anisotropy was measured on a microwell plate reader (Infinite M200Pro, Tecan), with a filter for excitation at 485 nm and emission at 535 nm. Dissociation constant (Kd) was calculated using the following equation on Prism 5 (GraphPad):

$$A = {A}_{f} + ( {A}_{b} - {A}_{f} ) \times \{ \frac{{[ L ]}_{T} + {K}_{D} + {[ P ]}_{T} - \sqrt{{( {[ L ]}_{T} + {K}_{D} + {[ P ]}_{T} )}^{2} - {{4 [ L ]}_{T} [ P ]}_{T}}}{{2 [ L ]}_{T}} \}$$

were A is anisotropy, A_{f} and A_{b} are the anisotropy values for free and bound ligand, respectively. [L]_{T} is the concentration of total fluorescent ligand and [P]_{T} the concentration of the protein.

For the measurements performed with calprotectin dimer, the dilution buffer did not contain CaCl₂ to avoid the formation of the tetramer. The rest of the protocol was identical.

Identified and synthethized peptides exhibited affinities to calprotectin in the nanomolar range (FIG. 1).

### Example 2: Identification of Linear Binding Sequence Based on Peptide 3 (RCPECVAFPMFQCHWYCG) Sufficient to Bind to Calprotectin

The peptides with defined pairs of cysteines bridged by chemical linkers were synthesized with two cysteines protected with Dpm groups and two with Mmt, instead of the previously used Trt. After the linear synthesis of the peptide and before the total cleavage, the resin was treated with 5 mL of TFA:TIS:DCM (1:5:94) in the fritted syringe for 8×2 min. The resin was washed 3 times with DCM and 3 times with DMF. 1.5 eq. of cyclization reagent and 4eq. of DIPEA in 4 mL of DMF were added and the reaction mixture was shaken for 1 h at room temperature. The reaction solution was removed and the resin was washed 3 times with DCM. Then, the resin was subjected to global deprotection with 90% TFA, 2.5% thioanisol, 2.5% H₂O, 2.5% 1.2-ethanedithiol, 2.5% phenol, for 6-8 h to assure that all the Dpm groups are removed. Ether purification and HPLC purification were performed as previously described.

Linear peptide 3-biotin was synthesized following the previous protocol, where the second Lys installed into the sequence was added as an Fmoc-Lys(Dde)-OH and the last amino acid was incorporated as Boc-Arg(Pbf)-OH. After the completion of the synthesis, the protecting group of Lys was removed with 2% hydrazine in DMF and biotin was incorporated at the de-protected amino group. Finally, the peptide was fully deprotected and removed from the resin.

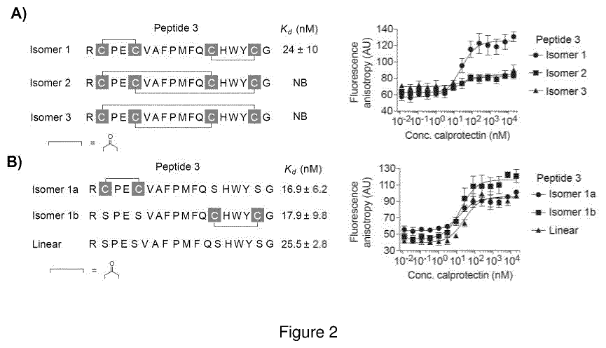

Subsequent fluorescence polarization experiments were performed according to example 1. The calprotectin binding affinity is impaired, if non-adjacent cysteines are cross-linked in peptide 3, indicating that peptide 3 binds to calprotectin in an elongated shape. In line with this observation a linear peptide with all cysteines replaced by serines exhibits similar affinity to calprotectin as isomers 1, 1a and 1b (FIG. 2).

### Example 3: Alanine Scan and Affinity Measurement by Surface Plasmon Resonance

The contribution of the different amino acids within linear peptide 3 was assessed using an alanine scan. For this reason the linear peptide was synthesized with selected replacements of individual amino acids with an alanine. The affinity of these linear peptides to calprotectin was then measured by fluorescence polarization (FP) and surface plasmon resonance (SPR).

In order to complement and validate the results obtained by FP, binding responses and kinetics of peptides with immobilized Calprotectin were analysed by SPR. The experiments were performed using a Biacore™ 8K instrument (GE Healthcare). Calprotectin (at 5 μg/mL) was dissolved in 10 mM acetate buffer (pH 5.0) and immobilized on a CM5 series S chip by standard amine coupling method in running buffer (10 mM HEPES pH 7.4, 150 mM NaCl, 2 mM CaCl₂, and 0.005% v/v Tween-20) at 25° C. Typical immobilization levels were 3000 resonance units (RUs). Reference cell was treated in the same way but without the injection of protein. A single concentration for each peptide (500 nM) was injected in running buffer (10 mM HEPES pH 7.4, 150 mM NaCl, 2 mM CaCl2, 0.005% v/v Tween-20 and 0.5% v/v DMSO) to measure the binding level. For the measurement of binding kinetics and dissociation constants, five serial dilutions (3-fold) of peptides were prepared in running buffer (with 0.5% DMSO) and analysed in single cycle kinetics mode with the contact and dissociation times of 90 s and 120 s, respectively.

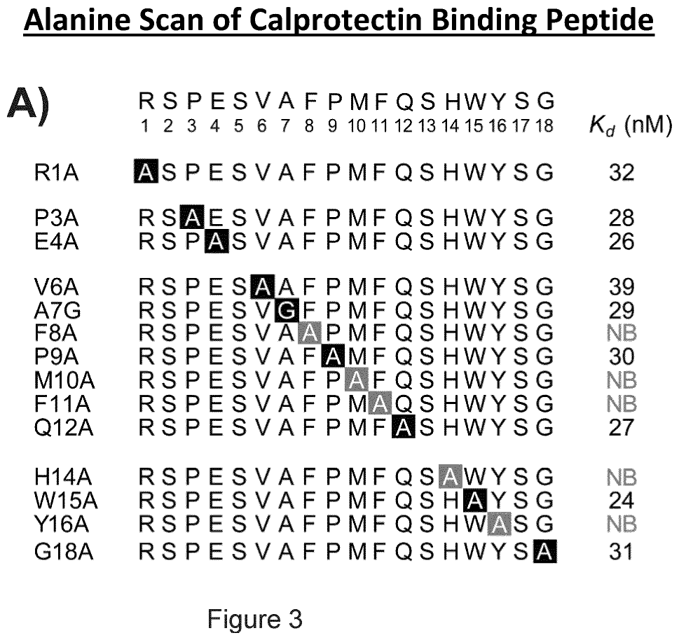

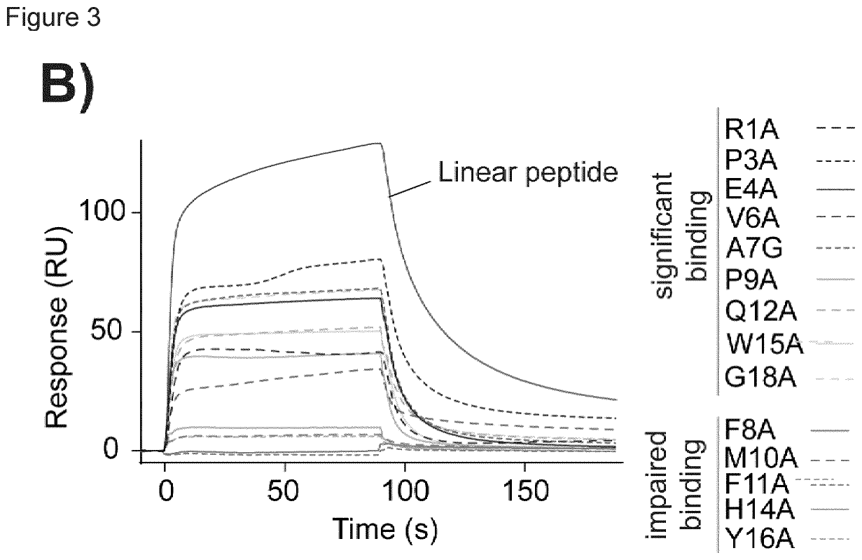

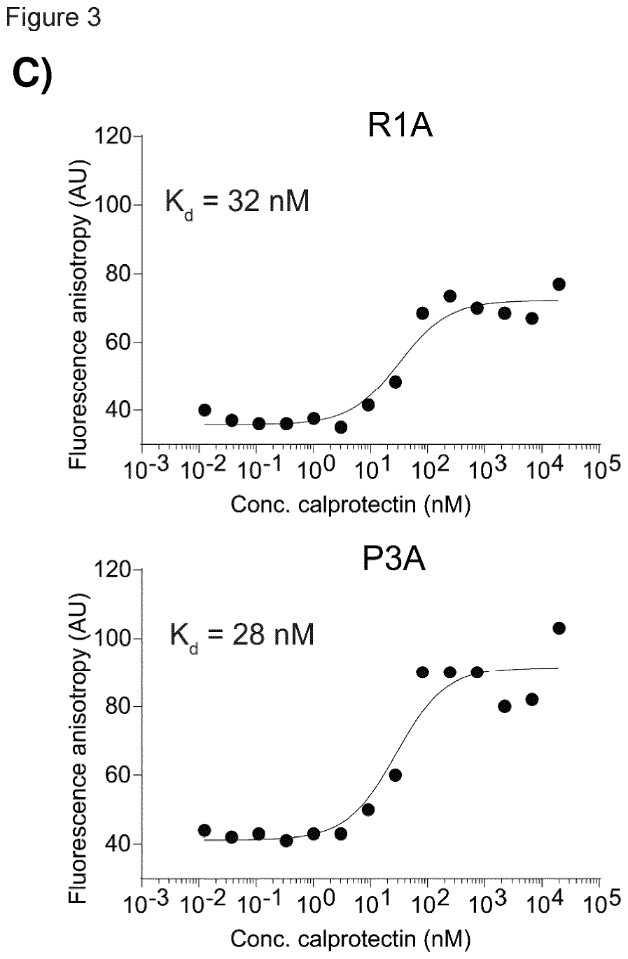

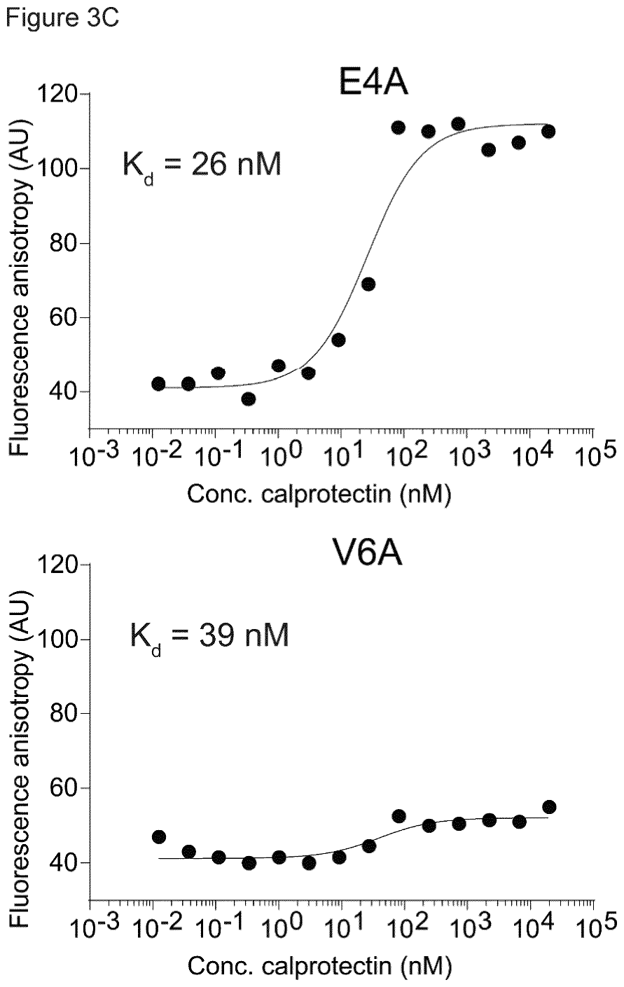

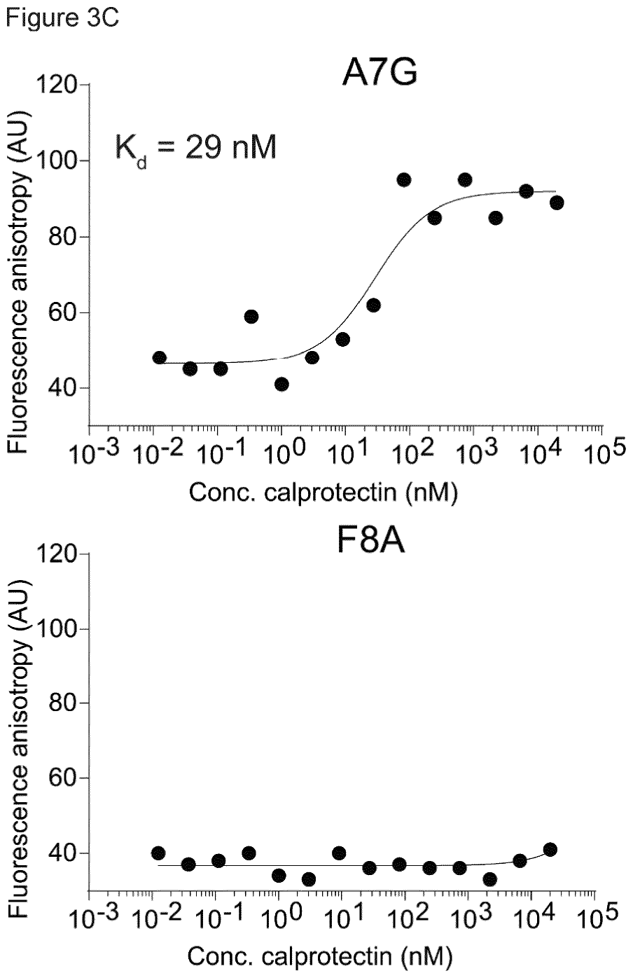

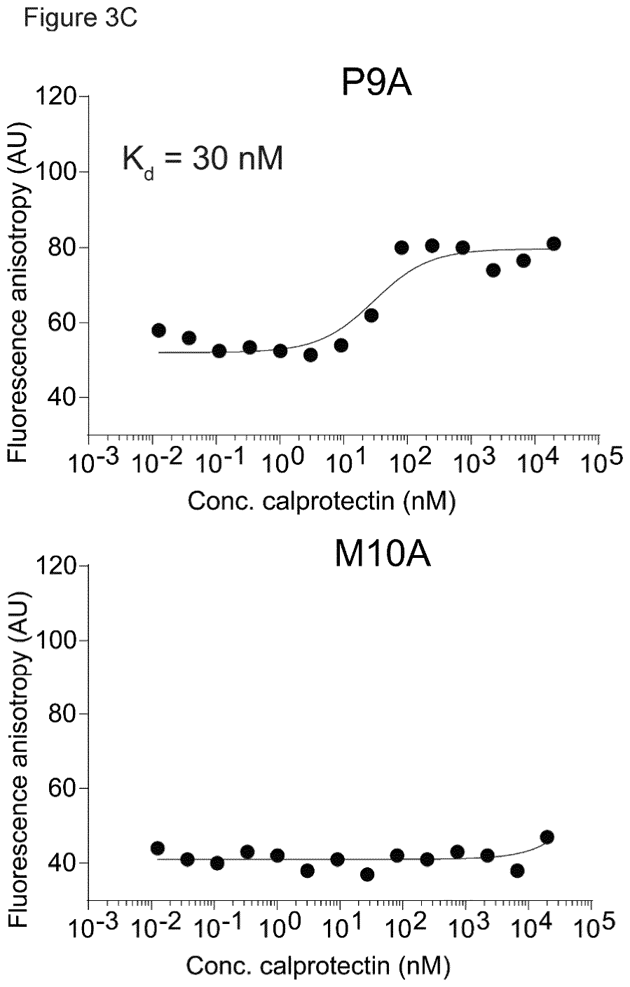

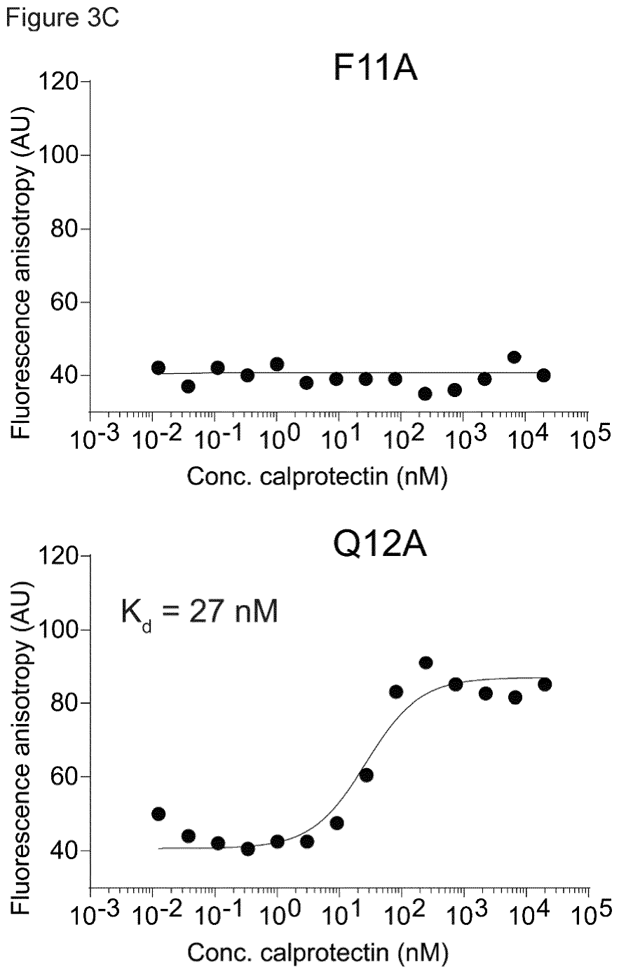

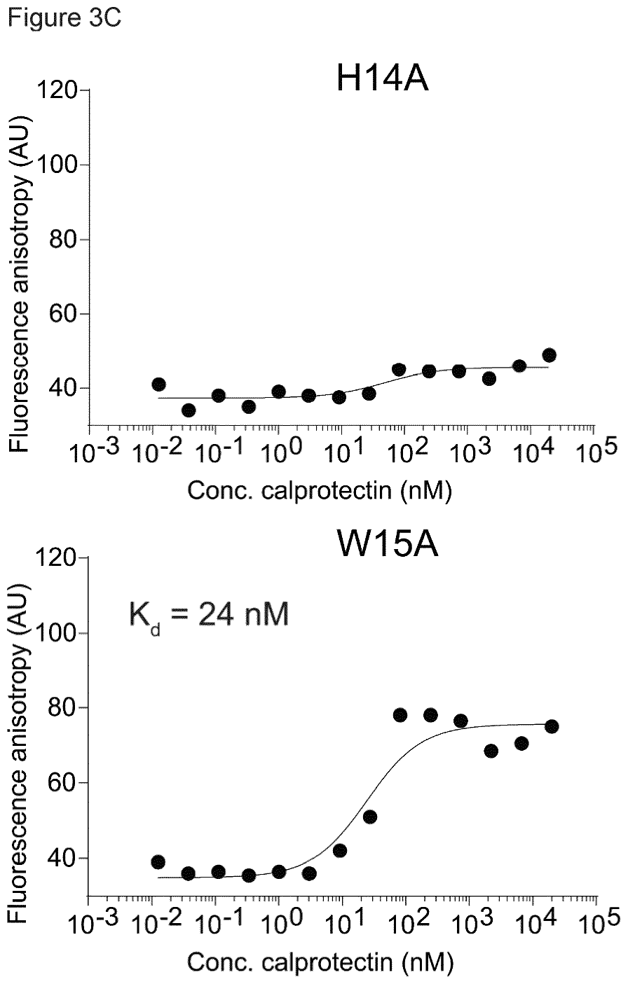

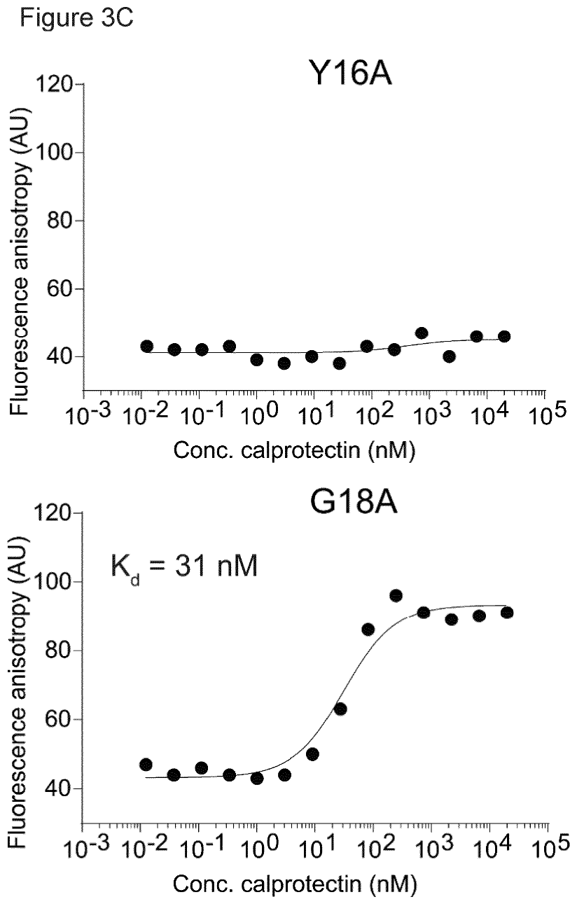

Both SPR and FP revealed that the amino acids that are mainly responsible for calprotectin affinity within linear peptide 3 are F8, M10, F11, H14 and Y16 (FIG. 3).

### Example 4: Co-Crystallization of Calprotectin and Linear Peptide 3

The fused calprotectin was expressed, cleaved and purified as described before. For crystallization purposes, cysteines of the fusion calprotectin were replaced by serines by site-directed mutagenesis to prevent disulfide bond formation. Prior to crystallization, the His-Tag and the linker sequence between S100A8 and S100A9 were cleaved by 3C precision protease in a 1:200 molar ratio over night at 4° C. The protein was subsequently concentrated using centrifugation devices with a 3-kDa cut-off to a final concentration of 11 mg/mL (440 μM) in 20 mM HEPES, 100 mM NaCl, 1 mM CaCl2, PH 7.4. Prior to crystallization, linear peptide 3 (6.2 mM, 14 equivalents) was incubated with the protein to allow the formation of the complex.

Crystals of the recombinant calprotectin with the peptide were grown at 18° C. employing the sitting drop vapor diffusion technique. Screening of crystallization conditions using an automated Mosquito crystal robot (SPT Labtech) and PACT Premier (Molecular Dimensions) yielded a crystal that appeared between day 3 and 5. The droplets contained 200 nL of protein solution and 100 nL of precipitant solution and were equilibrated against 100 μL of precipitant solution in a 96-well intelli-plate (Hampton Research). The best crystals grew in the condition containing 0.1 M MIB (Sodium malonate dibasic monohydrate, Imidazole, Boric acid), pH 6.0, 25% w/v PEG 1500, as the precipitant solution. The crystal was transferred to a cryogenic solution (25% glycerol) and flash-frozen in liquid nitrogen.

Data for calprotectin crystals in complex with peptide 3 was collected at the beamline PXIII of the Swiss Light Source at the Paul Scherrer Institute (SLS, Villigen, Switzerland) at a wavelength of 1.0 A. Raw data were processed with the program XDS. The structure was solved by molecular replacement using Phenix with the atomic coordinates of calprotectin (PDBID: 1XK4) as a search model. The peptide was manually modelled into the extra density using Coot. The structures were completed by iterative refinement in Phenix and model building in Coot, achieving a final model at 1.85 Å resolution. One copy of calprotectin (S100A8 and S100A9) and one copy of peptide constitute the asymmetric unit, while the calprotectin tetramer is formed by symmetry. Initial difference electron density maps revealed density consistent with the presence of 3 Ca(II) ions, 1 Ni(II) ion, 1 Na(I) ion and 1 K(I) ion. Molecular graphic figures were generated using PyMOL. The structure of the peptide with calprotectin shows the exact binding epitope of peptide three on calprotectin. Two peptides bind one calprotectin tetramer in an elongated form. The binding epitope involves Mrp8/Mrp14 of one calprotectin dimer as well as Mrp8 of the second dimer. This binding mode also illustrates that peptide 3 requires the calprotectin tetramer for binding (FIG. 4).

### Example 5: Sandwich ELISA with Calprotectin Binding Peptide

The wells of 96 well Immulon 4 HBX-Extra High Binding plates (Thermo Fisher Scientific) were coated by overnight incubation at 4° C. with 80 μL of a 0.5 μg/mL solution of anti-calprotectin rabbit polyclonal antibody in Na₂HCO₃ (0.2 M, pH 9.4). The plates were rinsed four times with 200 μL of washing buffer (25 mM Tris, 150 mM NaCl, 2 mM CaCl₂, PH 7.4, with 0.05% v/v Tween-20) per well. Then, all the wells were blocked by incubation with 300 μL of blocking buffer (25 mM Tris, 150 mM NaCl, pH 7.4, with 0.05% v/v Tween-20 and 3% w/v BSA) for 1 h at room temperature with shaking. After this time, the blocking buffer was removed and 80 μL of calprotectin was added in dilution buffer (wash buffer with 1% w/v BSA) for 1h at room temperature with shaking. The wells were washed 4 times with washing buffer. The peptide (linear peptide 3-biotin at 50 nM final concentration) was pre-mixed with Strep-HRP (4-fold less, Thermo Fisher Scientific) for 30 min and added to the wells for 2 h at room temperature with shaking. The solution was removed and the wells were washed 6times with washing buffer. 80 μL of TMB substrate (ready to use solution, SigmaAldrich) were added for 30 minutes and the reaction was stopped with 40 μL of sulfuric acid (2 M). The absorbance was read at 450 nm in a microwell plate reader (Infinite M200Pro, Tecan).

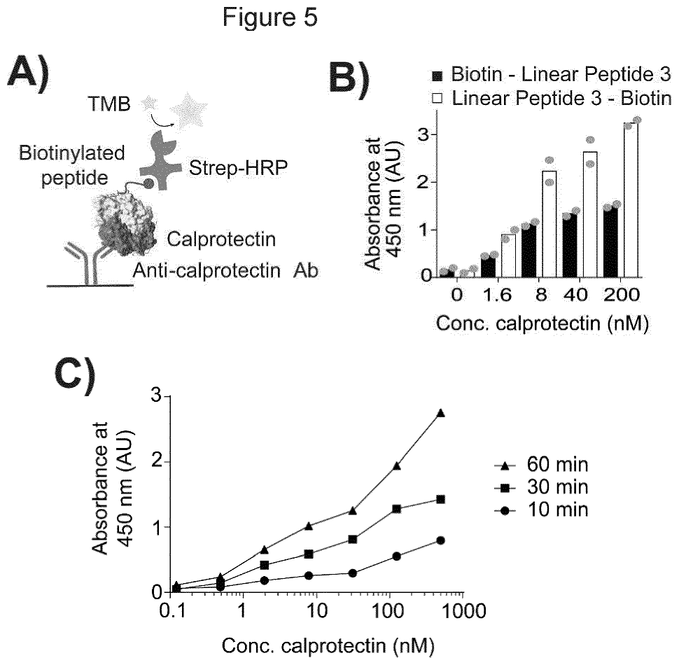

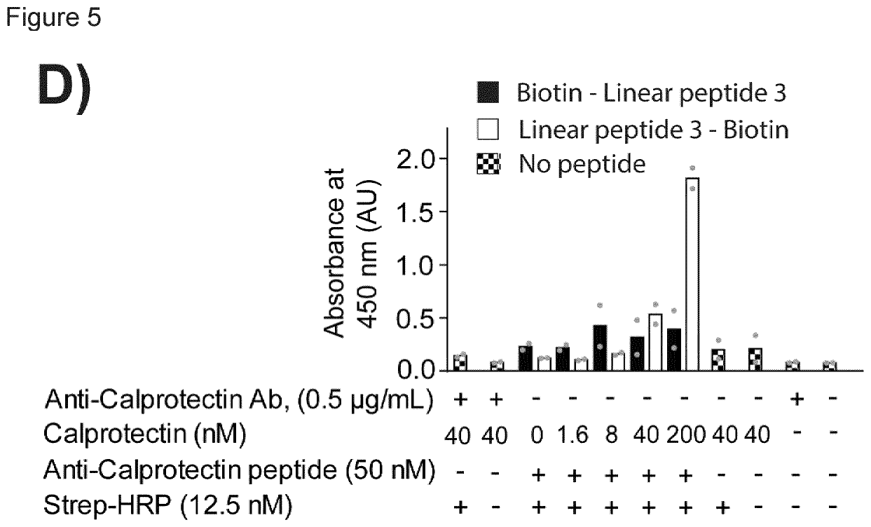

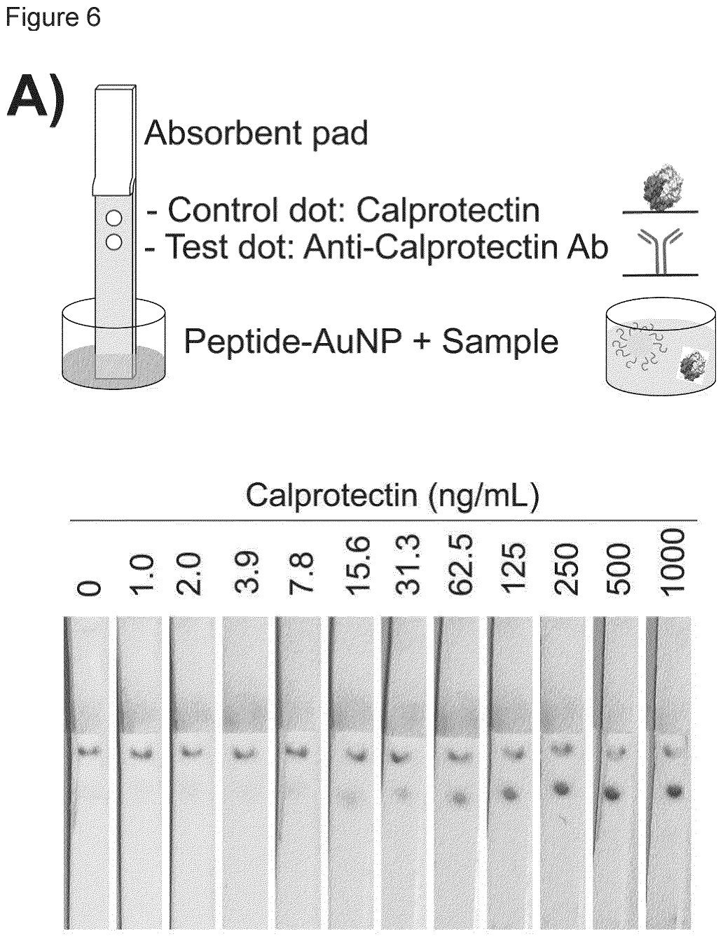

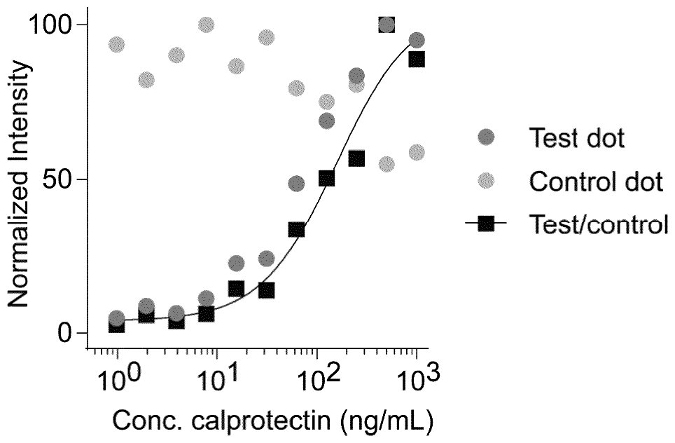

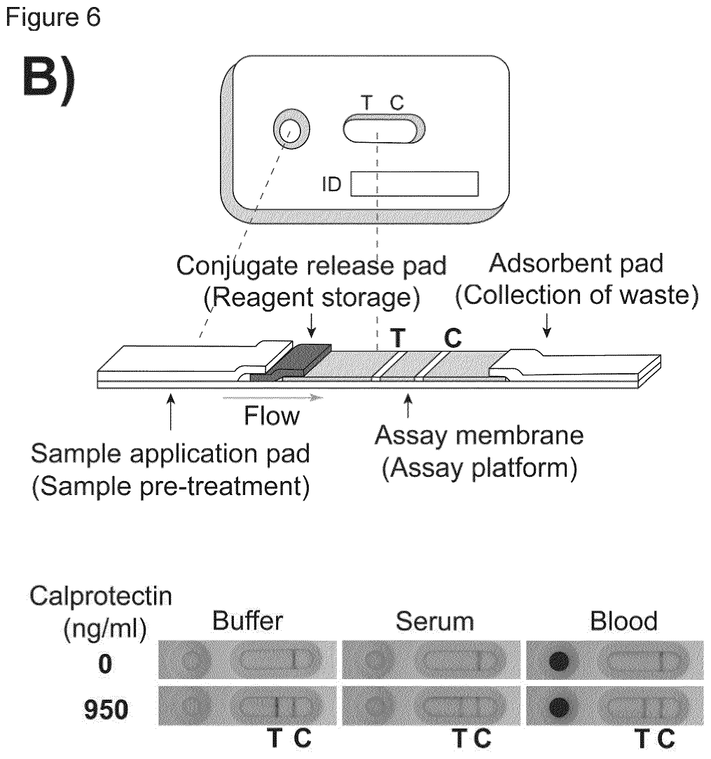

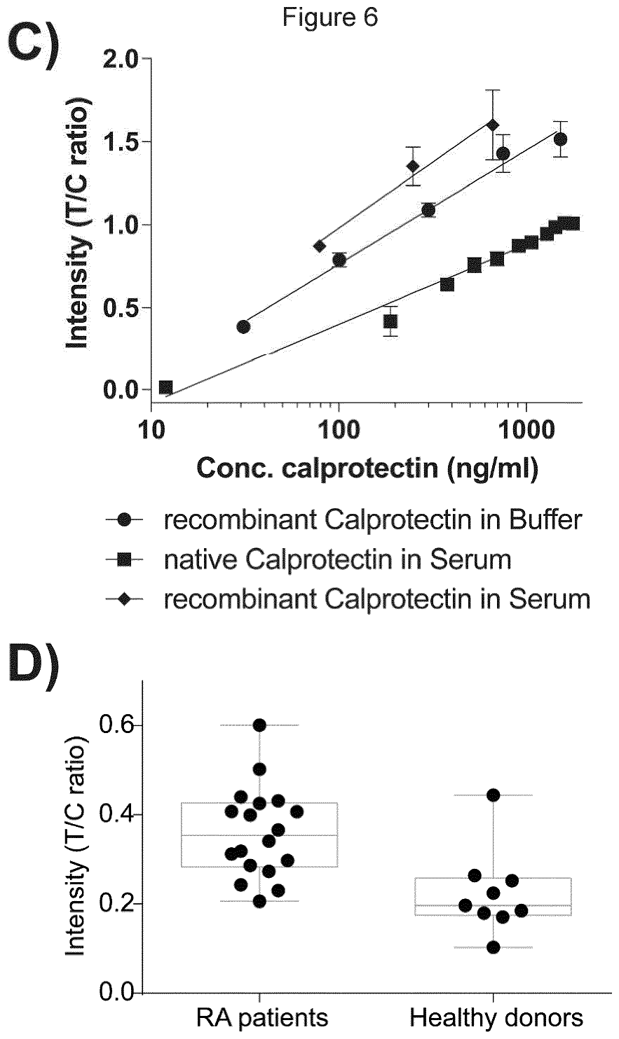

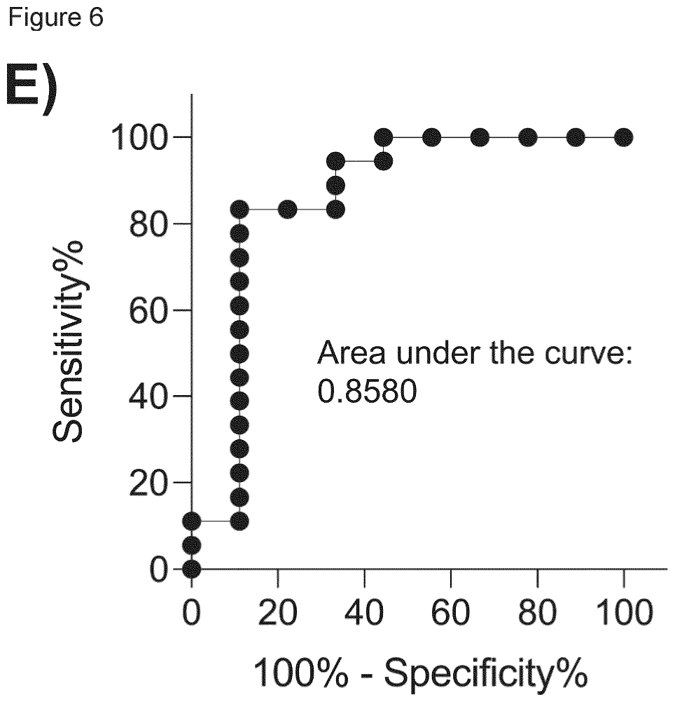

The ELISA results show that a C-terminal conjugation with biotin and Streptavidin-HRP is favorable over N-terminal conjugation, but in both set-ups a calprotectin concentration dependent ELISA signal can be measured. Elongated incubation times of 60 minutes of antibody captured calprotectin with the Strep-HRP biotinylated peptide complex may allow detection of calprotectin concentrations below 5 nM (FIG. 5).

### Example 6: Use of a Calprotectin Binding Peptide in a Lateral Flow Assay Set-up

Firstly, peptide-gold nanoparticles were prepared: linear peptide 3 biotinylated at the C-terminus and at a concentration of 1 M was incubated with streptavidin-coated gold nanoparticles (AuNPs). Free streptavidin sites were blocked by the addition of excess biotin. Peptide-AuNPs were concentrated by centrifugation and resuspended in conjugate resuspension buffer. These peptide-gold nanoparticles were used for the production of half-strip assays and lateral flow assays, respectively.

For half-strip assays a nitrocellulose membrane was stuck onto a sticky backing card together with an absorbent pad that overlapped the nitrocellulose membrane by 2 mm. Strips of 0.5 cm were cut using scissors. The anti-calprotectin antibody was applied as a dot to the test region 1.9 cm distant from the bottom of the nitrocellulose membrane. Recombinant calprotectin (B-RCAL) was applied as a dot to the test region 1.5 cm distant from the bottom of the nitrocellulose membrane. The half-strip was put vertically into a well of a flat-bottom 96-well plate containing 100 μL chase buffer, containing peptide-AuNPs and calprotectin. The half-strip was removed after 15 minutes. For a quantitative analysis, the intensity of the dots was measured using Image J. Curves were plotted using GraphPad Prism.

Lateral flow assays were produced by assembling the membrane and pads as described above, cut into strips of 0.5 cm width, and assembled into cassette housings. Samples containing calprotectin were applied to the LF strips. Blood was anticoagulated by EDTA and spiked with 9.5 μg/mL B-RCAL. Plasma was obtained of the calprotectin-spiked blood by centrifugation at 1′500×g and 4° C. for 15 min. Blood and plasma samples were diluted 10 times in chase buffer. Patient samples were obtained as serum fractions and were diluted 10 times with chase buffer before addition to the LF strip. To run the LFA, 80 μL of the premixed sample was applied to the SAP. After the sample solution was allowed to migrate through the membrane for 15 min at room temperature, the appearance of red lines at C and T positions indicated the detection of calprotectin. A negative result was indicated by the appearance of a red line only on the C line. For a quantitative analysis, the intensity was measured using a Quantum Blue® reader (3^{rd} generation). Curves were plotted using GraphPad Prism.

The prototype lateral flow assay was capable of measuring calprotectin concentrations quantitatively and in the current set-up is compatible with buffer, serum and capillary blood as matrix that require individual calibration curves. Moreover, an initial dataset of rheumatoid arthritis (RA) patient sera showed significantly elevated calprotectin concentrations compared to normal donors in this set-up.

### Example 7: Structure Activity Relationship (SAR) analysis of Peptide 3

The structure-activity relationship (SAR) analysis of Peptide 3 was assessed by synthesizing a large number of variants of Peptide 3 in which only one amino acid was altered at a time and the binding affinity was measured by surface plasmon resonance (SPR) and fluorescence polarization (FP).

In a first part, an L-alanine scan (or usually just termed alanine scan), a D-alanine scan, and a β-amino acid alanine scan were performed. The L-alanine scan was supposed to provide information about the importance of the side chain of each one of the 18 amino acids of Peptide 3 (RSPESVAFPMFQSHWYSG). The D-alanine scan was expected to tell if an inverted stereocenter at the alpha-carbon would be tolerated for the 18 different amino acids. In such mutations, the amino acid has two possibilities to retain the binding interaction, the first one being that it maintains the same orientation of the backbone atoms (amino group, carbonyl, alpha-carbon) and changes the position and orientation of the side chain, or the second one being that the amino acid keeps the side chain interaction identical but accepts a larger change in the interaction of the backbone, which also affects neighboring amino acids. Results of the D-alanine scan provide valuable information mostly for amino acid positions where the L-alanine is binding, but not for positions where mutation to L-alanine renders the peptide inactive (as in such a case, it would not be clear if loss of activity is due to side chain deletion or change of stereocenter). The β-amino acid alanine scan was performed to study if changes in the backbone (addition of one carbon atom) would be tolerated for the 18 positions of Peptide 3. This change has obviously consequences also for the neighboring amino acids. As for the D-alanine scan, the results of the β-amino acid alanine scan were only much conclusive in case the L-amino acid mutation was active.

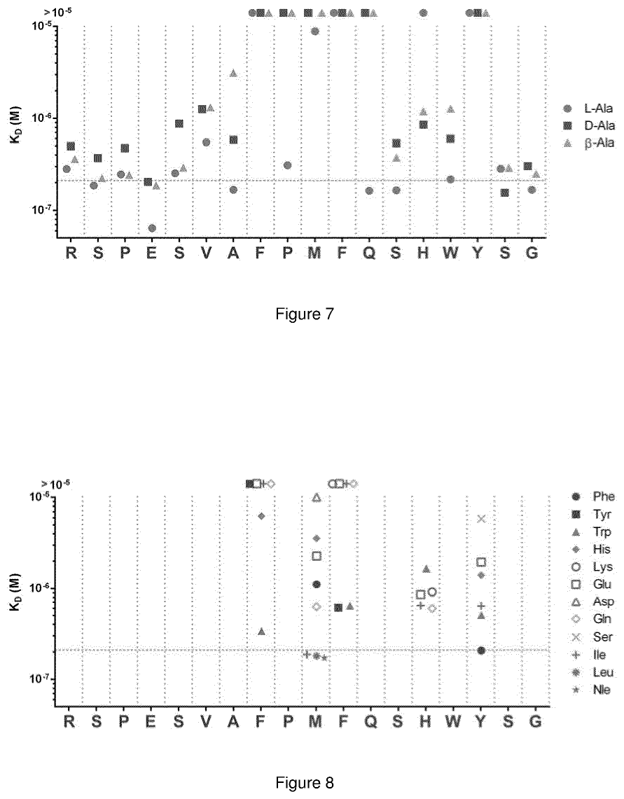

The results of the three “scans” are shown in FIG. 7. The results show that the side chains of five amino acids were most critical for the binding of Peptide 3 (F8, M10, F11, H14, Y16), meaning that their side chains most likely contributed most of the binding energy of Peptide 3 (results of the L-alanine scan). The results was overall in line with the data of the phage display selections, where these positions were most conserved in peptides that were enriched for calprotectin binding. Surprisingly, the P9 amino acid was not among the five amino acids for which the side chain plays an important role, despite the strong conservation of this position in the phage display selections (nearly all phage display-selected peptides contained a proline in this position). It could be that L-alanine is well accepted due to the similarity to L-proline, and that the side chain in this position contributes also much to the binding affinity of Peptide 3.

For the 13 amino acids of Peptide 3 where mutation to L-alanine was tolerated and led to only a minimal loss in binding affinity, two amino acids showed major losses in binding if mutated to D-alanine or β-alanine, being P9 and Q13. As discussed above, P9 might tolerate L-alanine due to the similarity to L-proline, but the position definitively does not accepted changes in the backbone or the a-carbon configuration. For Q13, the result indicated that the side chain is not so critical for this position, but that the backbone region was key, as for example to ideally space or orient the neighboring amino acids. For both positions, P9 and Q13, it is thus important that they are occupied by amino acids that are a-amino acids and that have an L-configuration.

A broader side chain scan at the five amino acid positons that appeared to have important side chain interreactions based on the L-alanine scan showed how much each side chain can be changed, and revealed some positions where the side chain cannot be changed much (FIG. 8). Phe8 can be modified with Trp without losing much of the binding, yet it cannot be replaced with other aromatic amino acids such as Tyr or His. In the case of Tyr, it is mostly due to a steric clash of calprotectin with the tyrosine alcohol at para position. Met10 could be replaced with other aliphatic amino acids (IIe, Leu, and NIe) without any loss of binding. This result encourages its future substitution to avoid methionine oxidation. Phe11 tolerated both Tyr and Trp aromatic side chains. His14 and Tyr16 tolerated a wide variety of different amino acids with only a small decrease in affinity. Interestingly, the mutation of Tyr16 to Phe did not lead to a loss of binding, which suggests a non-critical role of the alcohol in Tyr16.

1. A peptide capable of binding calprotectin, the peptide comprising the sequence ΦZΨΣXΘΘΘΩ, wherein
ϕ is L-phenylalanine, L-tryptophan or an analogue thereof,
X and Z represent an α-amino acid with L-configuration,
Ψ represents a nonpolar, aliphatic L-amino acid,
Σ is L-tyrosine, L-tryptophan, L-phenylalanine or an aromatic L-amino acid,
Θ represents an a-amino acid,
Ω represents a hydrophobic L-amino acid and
wherein ϕ at each occurrence, Z at each occurrence, X at each occurrence, Ψ at each occurrence, Σ at each occurrence, Θ at each occurrence and Ω at each occurrence, are selected independently from each other.

2. The peptide according to claim 1, wherein Ψ is L-methionine, L-leucine, L-isoleucine or L-norleucine and/or Z is L-proline or L-alanine.

3. The peptide according to claim 1 or 2, wherein at least one of the following conditions is fulfilled: ϕ is L-phenylalanine or a derivative thereof, Z is L-proline or a derivative thereof, Ψ is L-methionine or a derivative thereof, Σ is L-phenylalanine or a derivative thereof and/or Ω is L-tyrosine or a derivative thereof.

4. The peptide according to any of claims 1 to 3, wherein the peptide comprises the sequence FPLFQΘΘΘY. FPIFQΘΘΘY, FP(NIe)FQΘΘΘY, FPLFQΘΘΘF, WPLFQΘΘΘY, FPIFQΘΘΘF, FP(NIe)FQΘΘΘF, WPIFQΘΘΘY, WP(NIe)FQΘΘΘY, WPLFQΘΘΘF, WPIFQΘΘΘF, WP(NIe)FQΘΘΘF or FZMFXΘHΘY.

5. The peptide according to any of claims 1 to 4, wherein the peptide consists of 9 to 30 amino acids.

6. The peptide according to any of claims 1 to 5 having the sequence RCPECVAFPMFQCHWYCG or RSPESVAFPMFQSHWYSG.

7. The peptide according to claim 1 comprising the sequence CTQSPCPLYDSHQCSCK, VCPCPLFRAHGCSRFSCQ, CQCPWDLFSQHSLSDCCD, WCTQSPCPLYDSHQCSCK, TCPLNRTQCPLYACTTCP, GCDLAHQPCPLYKCTKCP, VCQQTASRCPVWECQRCP, ACRTCPLFTCPSCG, or SCQCPWDLFSQHSLSDCCD.

8. The peptide according to any of claims 1 to 7, wherein the equilibrium dissociation constant K_{D} of the peptide for calprotectin is between 1 pM and 750 nM.

9. A method for detecting calprotectin in a sample, the method comprising
d) providing a sample comprising calprotectin;
e) contacting the sample with the peptide according to any of claims 1 to 8 and allowing the peptide form a complex with the calprotectin;
f) detecting the complex formed in step b).

10. The method according to claim 9, wherein the peptide is detectably labelled, preferably with a nanoparticle, streptavidin, biotin, a fluorescent marker, a luminescent marker or an enzyme.

11. A kit for detecting calprotectin in a sample, the kit comprising the peptide according to any of claims 1 to 8.

12. A method of purifying calprotectin, the method comprising purifying the calprotectin using a peptide according to any of claims 1 to 8.

13. The method according to claim 9, wherein the peptide is immobilized on a stationary phase.

14. A pharmaceutical composition comprising the peptide according to any of claims 1 to 8.

15. Use of a peptide according to any of claims 1 to 8 for tagging a protein of interest.


SEQUENCE LISTING (24 sequences)

SEQ ID NO: 1 - protein, 9 aa, synthetic construct
  VARIANT 7..9: note = X represents can be any alpha amino acid
FPLFQXXXY

SEQ ID NO: 2 - protein, 9 aa, synthetic construct
  VARIANT 7..9: note = X represent can be any alpha amino acid
FPIFQXXXY

SEQ ID NO: 3 - protein, 9 aa, synthetic construct
  VARIANT 3: note = Nle
  VARIANT 7..9: note = X can be any alpha amino acid
FPXFQXXXY

SEQ ID NO: 4 - protein, 9 aa, synthetic construct
  VARIANT 7..9: note = X can be any an alpha amino acid
FPLFQXXXF

SEQ ID NO: 5 - protein, 9 aa, synthetic construct
  VARIANT 7..9: note = X can be any an alpha amino acid
WPLFQXXXY

SEQ ID NO: 6 - protein, 9 aa, synthetic construct
  VARIANT 7..9: note = X can be any an alpha amino acid
FPIFQXXXF

SEQ ID NO: 7 - protein, 9 aa, synthetic construct
  VARIANT 3: note = Nle
  VARIANT 7..9: note = X can be any an alpha amino acid
FPXFQXXXF

SEQ ID NO: 8 - protein, 9 aa, synthetic construct
  VARIANT 7..9: note = X can be any an alpha amino acid
WPIFQXXXY

SEQ ID NO: 9 - protein, 9 aa, synthetic construct
  VARIANT 3: note = Nle
  VARIANT 7: note = X can be any alpha amino acid with L-configuration
WPXFQXXXY

SEQ ID NO: 10 - protein, 9 aa, synthetic construct
  VARIANT 7..9: note = X can be any an alpha amino acid
WPLFQXXXF

SEQ ID NO: 11 - protein, 9 aa, synthetic construct
  VARIANT 7..9: note = X can be any an alpha amino acid
WPIFQXXXF

SEQ ID NO: 12 - protein, 9 aa, synthetic construct
  VARIANT 3: note = Nle
  VARIANT 7..9: note = X can be any an alpha amino acid
WPXFQXXXF

SEQ ID NO: 13 - protein, 9 aa, synthetic construct
  VARIANT 7..9: note = X can be any an alpha amino acid
FXMFXXHXY

SEQ ID NO: 14 - protein, 18 aa, synthetic construct
RCPECVAFPMFQCHWYCG

SEQ ID NO: 15 - protein, 18 aa, synthetic construct
RSPESVAFPMFQSHWYSG

SEQ ID NO: 16 - protein, 17 aa, synthetic construct
CTQSPCPLYDSHQCSCK

SEQ ID NO: 17 - protein, 18 aa, synthetic construct
VCPCPLFRAHGCSRFSCQ

SEQ ID NO: 18 - protein, 18 aa, synthetic construct
CQCPWDLFSQHSLSDCCD

SEQ ID NO: 19 - protein, 18 aa, synthetic construct
WCTQSPCPLYDSHQCSCK

SEQ ID NO: 20 - protein, 18 aa, synthetic construct
TCPLNRTQCPLYACTTCP

SEQ ID NO: 21 - protein, 18 aa, synthetic construct
GCDLAHQPCPLYKCTKCP

SEQ ID NO: 22 - protein, 18 aa, synthetic construct
VCQQTASRCPVWECQRCP

SEQ ID NO: 23 - protein, 14 aa, synthetic construct
ACRTCPLFTCPSCG

SEQ ID NO: 24 - protein, 19 aa, synthetic construct
SCQCPWDLFSQHSLSDCCD